(19) United States
(12) Patent Application Publication
(10) Pub. No.: US 2024/0066174 A1
(43) Pub. Date: Feb. 29, 2024

(54) DIFFUSER SECURED TO CONTAINER DRAWING OIL FRAGRANCE INTO DIFFUSER ELEMENT VIA CAPILLARY ACTION

(71) Applicant: Ulta-Lit Tree Company, Glenview, IL (US)

(72) Inventors: John Christopher Krolopp, Glenview, IL (US); John DeCosmo, Glenview, IL (US); Gregory James Foster, Glenview, IL (US)

(21) Appl. No.: 18/127,221

(22) Filed: Mar. 28, 2023

Related U.S. Application Data
(63) Continuation of application No. PCT/US23/16461, filed on Mar. 27, 2023.
(60) Provisional application No. 63/324,608, filed on Mar. 28, 2022.
(60) Provisional application No. 63/416,402, filed on Oct. 14, 2022.

(51) Int. Cl. A61L 9/12

(52) U.S. Cl. CPC A61L 9/127; A61L 2209/134; A61L 2209/15

(57) ABSTRACT

A diffuser made of thermoform molded pulp in a desired shape, such as a hanging ornament, having features to secure a wicking part of the diffuser to a rounded bottle opening filled with an oil fragrance. To insert the diffuser, a cap is unscrewed from a bottle filled with oil fragrance, and the wicking part is inserted through the opening of the bottle until it rests on the interior bottom of the bottle. A rotatable capping feature is featured at the top of the wicking part, which is closed during insertion of the wicking part to prevent accidental spillage and loss oil due to evaporation. The wicking element draws the oil fragrance into the ornamental diffusing part by capillary action.

### CROSS-REFERENCE TO RELATED APPLICATIONS

This application is a continuation of International Application No. PCT/US23/16461, filed Mar. 27, 2023, which claims the benefit of, and priority to, U.S. Provisional Patent Application No. 63/324,608, filed Mar. 28, 2022 and U.S. Provisional Patent Application No. 63/416,402, filed Oct. 14, 2022, each of which is hereby incorporated by reference herein in its entirety.

### FIELD OF THE INVENTION

The present disclosure relates to passive diffuser elements that cause a volatile liquid to be evaporated into the atmosphere.

### BACKGROUND

Passive diffusers, which do not require any powered assistance to transform a liquid into evaporated gas that disseminates into a surrounding atmosphere or environment, come in many forms. One common example is an aromatherapy reed diffuser in which the user inserts elongated reeds into a scented liquid, which is contained within an open glass container or reservoir. Each reed takes up the liquid by capillary action and diffuses it into the atmosphere by evaporation. However, to insert the reeds, the glass container must be left uncapped, allowing evaporation to escape through the open container, and if tipped over, its liquid contents will spill out, nullifying or greatly diminishing any further useful diffusing effect from what little liquid may remain in the container. Moreover, each reed has a relatively small surface area, constrained by its diameter and length. To adjust the strength of the diffused scent, the user is instructed to add or subtract reeds. Moreover, the rate of release of each reed is constrained by the small cross-sectional end plus whatever length of the reed is submerged in the liquid, and the diffuser is generally suitable for small rooms like a bathroom. As this liquid level drops, this rate of release decreases, so even if all reeds are used, over time, the strength of the scent release will diminish before the liquid is completely removed from the container, both by capillary action through the reeds and evaporation through the open container.

In addition to spillage risk, unnecessary evaporation losses, and diminishing rate of scent release, another problem that plagues passive diffusers is that they are not susceptible to being hung, like an ornament. Certainly, the reed diffusers mentioned above cannot be hung without some external contraption to hold the container. Many are simply too heavy to be hung on a relatively flimsy or flexible structure, like a tree branch. A scented fragrance stick sold under the trademark SCENTSICLES is capable of being hung, but these sticks are pre-scented and have a form factor that is in the shape of a thin rod made of tightly rolled-up paper. They do not draw up any scent through capillary action, so once they are removed from their container, they begin to diffuse their scent into the atmosphere, which decreases over time until their pre-soaked scent load has been exhausted. The rod-shape does not inspire or evoke any particular theme or holiday, even though they are typically marketed as Christmas ornaments. Thus, these type of scented hanging ornaments cannot benefit from capillary action and are not amenable to thematic, imaginative, or wholly fanciful designs or shapes due to their construction of using rolled-up paper to form a rod-shaped “ornament.”

Furthermore, conventional passive diffusers have static elements that, once inserted into a reservoir containing the liquid to be diffused, remain in their inserted location (unless moved by the user). Thus, for example, any liquid that remains in the reservoir that is not lost due to evaporation through an opening in the container, might not be accessible by the diffuser element if it not placed in the location where the liquid can pool or collect as it draws down toward the bottom of the container. Leaving liquid in the container that is not sucked up by capillary action into the diffuser element(s) unnecessarily shortens the useful life of the diffuser. Even a small amount of liquid can translate into days of additional evaporative release into the surrounding environment.

A need exists for an improved device that does not suffer from any one or more of these and other drawbacks. A need also exists for an improved device that overcomes all of the aforementioned drawbacks, by themselves and in addition to others. The present disclosure address these and other needs.

### SUMMARY

According to an aspect of the present disclosure, an apparatus for diffusing a volatile substance to an ambient environment is disclosed.

The apparatus includes: an elongated wick portion having a free end configured to be inserted into a container containing a volatile substance that is drawn through capillary action into the elongated wick portion; an emanating portion having an exposed surface from which the volatile substance evaporates into the ambient environment; a plug portion having a hinge member coupled to the elongated wick portion, the plug portion being configured to rotate about the hinge member to form, together with the elongated wick portion, a plug that is configured to be inserted into an opening of the container to suppress release of the volatile substance due to evaporation via the opening and to maximize dissemination of the volatile substance through capillary action from the elongated wick portion to the emanating portion.

The apparatus can be combined with the container, the container can have a bottle shape and a screw cap over the opening and a shoulder between the opening and a body of the bottle, and the volatile substance can include a fragrance oil. The combination of the apparatus and the container can have a weight not exceeding two ounces. The protruding member can be configured to be inserted into the opening and past the shoulder until it rests against the shoulder inside the container.

The elongated wick portion can have a semi-circular cross-section. The plug portion can have a semi-circular cross-section such that when the plug portion is joined with the elongated wick portion, the plug has a generally circular cross-section substantially closes the opening to the container.

The apparatus can further include a first protruding member on the elongated wick portion configured to couple the apparatus to a container containing the volatile substance without the apparatus and container uncoupling under a weight of the container, and a second protruding member opposite the first protruding member, each of the first protruding member and the second protruding member protruding away from the elongated wick portion and having a tapered portion that tapers toward the elongated wick portion toward the free end.

Each of the first protruding member and the second protruding member can be flexible to squeeze into the opening of the container until they expand once moved beyond the opening of the container.

The free end of the elongated wick portion can have one or more flexible legs, each of the flexible legs being configured to bend and to extend toward sidewalls of an interior of the body of the container in response to the elongated wick portion being inserted through the opening until the free end contacts a bottom interior surface of the container and each of the plurality of flexible legs splay out away from one another.

The apparatus can further include a hole in the emanating portion configured to receive therethrough a hanging element for hanging the apparatus as an ornament. The emanating portion can have a dimension across its surface along a cross-section of the emanating part, the dimension being at least 10% greater than a width of the emanating portion along the same cross-section.

The elongated wick portion and the emanating portion can form a unitary, integral piece composed of a thermoform molded pulp. The elongated wick portion, the emanating portion, and the plug portion can be formed as a unitary, integral piece from a thermoform molded pulp introduce to a mold. The thermoformed molded pulp can be of Type 3. The volatile substance can comprise an oil-based fragrance.

According to some implementations of the present disclosure, an apparatus for diffusing a volatile substance to an ambient environment is disclosed. The apparatus includes a plug, a wick portion, and an emanating portion. The plug is configured to be at least partially inserted into an opening of a container containing a volatile substance to aid in at least partially inhibiting the volatile substance from flowing out of the container via the opening. The wick portion comprises thermoform molded pulp configured to cause the volatile substance to be drawn up into the wick portion through capillary action. The emanating portion comprises thermoform molded pulp and coupled to the wick portion such that the volatile substance is drawn up into the emanating portion through capillary action, the emanating portion having an exposed surface configured to aid in diffusing the volatile substance into the ambient environment at an average rate of release that is greater than about 0.25 grams of the volatile substance per day over 30 days.

The thermoformed molded pulp can comprise sugarcane bagasse. The volatile substance can include an oil-based fragrance. The oil-based fragrance can comprise 100% oil. In some implementations, the average rate of release is greater than about 0.35 grams of the oil-based fragrance per day over 30 days. In some implementations, the emanating portion is configured to diffuse at least 0.1 grams of the oil-based fragrance into the ambient environment each day for 30 days. The in some implementations, the emanating portion is configured to diffuse at least 0.4 grams of the oil-based fragrance into the ambient environment each day during a first week, at least 0.3 grams of the oil-based fragrance into the ambient environment each day during a first week that is immediately subsequent to the first week, at least 0.2 grams of the oil-based fragrance into the ambient environment each day during a third week that is immediately subsequent to the second week, and at least 0.1 grams of the oil-based fragrance into the ambient environment each day during a fourth week that is immediately subsequent to the third week.

In some implementations, the emanating portion is configured to have an average rate of release of about 0.6 grams/day of the oil-based fragrance during a first week, an average rate of release of about 0.4 grams/day of the oil-based fragrance during a second week that is immediately subsequent to the first week, an average rate of release of about 0.25 grams/day of the oil-based fragrance during a third week that is immediately subsequent to the second week, and an average rate of release of about 0.2 grams/day of the oil-based fragrance during a fourth week that is immediately subsequent to the third week.

In some implementations, the plug and the wick portion are unitary and/or monolithic and the plug includes a first plug portion and a second plug portion that are moveable relative to one another. The plug includes a first plug portion and a second plug portion that are moveable relative to one another. The wick portion can include a hinge configured to permit the first plug portion and the second plug portion to move relative to one another between an open position and a closed position, wherein the closed position, the first plug portion and the second plug portion are configured to inserted into the opening of the container.

The first plug portion can include a first sealing protrusion configured to engage the container responsive to the plug being inserted into the container to aid in suppressing release of the volatile substance via the opening of the container. The second plug portion can include a second sealing protrusion configured to engage the container responsive to the plug being inserted into the container and aid in suppressing release of the volatile substance via the opening of the container. The first sealing protrusion and the second sealing protrusion can have a tear-drop shape. The container can have a bottle shape and a screw cap over the opening and a shoulder between the opening and a body of the bottle, and the first sealing protrusion and the second sealing protrusion can be configured to be inserted into the opening and past the shoulder until it rests against the shoulder inside the container.

The wick portion can further include a first tongue coupled to the first plug portion and a second tongue coupled to the second plug portion. In the closed position, the first tongue is coupled to the second tongue. The first tongue can include a plurality of apertures and the second tongue includes a plurality of protrusions, wherein in the closed position, the plurality of protrusions engage corresponding ones of the plurality of apertures to aid in coupling the first tongue and the second tongue and inhibiting movement of the first plug portion and the second plug portion relative to one another. The plurality of apertures in the first tongue can each have a diameter of about 4 mm.

The emanating portion can include a hole configured to receive therethrough a hanging element for hanging the apparatus as an ornament. The emanating portion can have a generally triangular shape. The emanating portion can include a plurality of grooves extending along a length thereof configured to aid in diffusing the volatile substance drawn into the wick portion into the ambient environment. The first plug portion and the second plug portion are configured to form a cavity therebetween responsive to the wick portion being in the closed position.

The apparatus can have a first length along its longitudinal axis in the open position and a second length along its axis in the closed position, wherein the second length is less than the first length. The second length can be between about 50% and about 75% of the first length.

The wick portion and the emanating portion form a unitary, integral piece composed of a thermoform molded pulp. The thermoform molded pulp can be of Type 3. The wick portion and the emanating portion can be formed as a unitary, integral piece from a thermoform molded pulp introduced to a mold.

The apparatus can be combined with the container, the container having a bottle shape and a removable screw cap over the opening, the volatile substance including a fragrance oil, the combination of the apparatus and the container having a weight not exceeding two ounces. The emanating portion can have a width that is at least 3 times greater than a diameter of the opening of the container.

The emanating portion can be configured such that the average rate of release is greater than about 2.5 grams of the volatile substance per week over 30 days. The emanating portion can be configured such that the average rate of release is between about 2.5 grams and about 3 grams of the volatile substance per week over 30 days.

The apparatus can be combined with a base including an aperture configured to receive a portion of the container therein. The base can include a plurality of protrusions configured to engage a surface of the container to couple the base to the container via an interference fit. The wick portion, the emanating portion, and the base can comprise a thermoform molded pulp. The thermoform molded pulp can be of Type 3.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

The foregoing and other advantages of the invention will become apparent upon reading the following detailed description and upon reference to the drawings.

FIG. 1 is a front perspective view of an apparatus for diffusing a volatile substance to an ambient environment and a bottle with a cap containing the volatile substance;

FIG. 2 is a cut-away of a rear perspective view of the apparatus and bottle shown in FIG. 1 with a plug portion ready to be rotated to form a plug;

FIG. 3 is a cut-away of the apparatus and bottle shown in FIG. 2 with the plug portion rotated into a closed position to form a plug prior to insertion into an opening of the bottle;

FIG. 4 is a cut-away of the apparatus and bottle shown in FIG. 3 with the elongated wick portion partially inserted into the opening of the bottle so that a free end of the wick portion begins to submerge into the volatile substance;

FIG. 5 is a cut-away of the apparatus and bottle shown in FIG. 4 with flexible protruding members being squeezed together against the wick portion as they pass through the opening and neck of the bottle;

FIG. 6 is a cut-away of the apparatus and bottle shown in FIG. 5 with the wick portion fully inserted into the bottle, with legs at the free end splayed out so that they reach the interior edges of the bottom surface of the bottle, and the protruding members have extended back into their original position to support the shoulder of the neck of the bottle so that the apparatus can be hung, such as an ornament, with the plug portion plugging the opening inside the neck to avoid spillage or evaporation of the volatile substance through the opening;

FIGS. 7A, 7B, and 7C illustrate the capillary action by which the volatile substance in the bottle is drawn up through the wick portion and then into the emanating portion throughout its surface where the volatile substance is evaporated into the surrounding atmosphere, e.g., as a fragrance;

FIG. 8A is a close-up of a cut-away of the bottle showing part of the wick portion inserted partially but not fully therein, with the protruding members beginning to deform as they are squeezed through the neck of the bottle;

FIG. 8B is a cross-sectional view of the bottle and wick portion shown in FIG. 8A;

FIG. 9A is a close-up of a cut-away of the bottle showing the legs of the wick portion at its free end beginning to splay outwards, while protruding members have cleared the neck of the bottle and return to their original, respective positions;

FIG. 9B is a cross-sectional view of the bottle and wick portion shown in FIG. 9A;

FIG. 10A is a close-up of a cut-away of the bottle showing the legs of the wick portion at its free end fully splayed apart, while protruding members have cleared the neck of the bottle and rest under the shoulder of the neck to support the weight of the bottle, such as when the apparatus is hung, and the plug portion is closed to form a plug and is inserted into the neck of the bottle;

FIG. 10B is a cross-sectional view of the bottle, wick portion, and closed plug portion shown in FIG. 10A;

FIG. 11 is a cross-sectional view taken from the lines 11-11 shown in FIG. 7A to illustrate the increased surface area available for evaporating the volatile substance compared to the width of the emanating portion;

FIG. 12 is a front perspective view of an apparatus for diffusing a volatile substance to an ambient environment, according to some implementations of the present disclosure;

FIG. 13 is a cross-sectional view of an emanating portion of the apparatus of FIG. 12, according to some implementations of the present disclosure;

FIG. 14A is a front perspective view of the apparatus of FIG. 12 in an open position, according to some implementations of the present disclosure;

FIG. 14B is a front perspective view of the apparatus transitioning from the opening position towards a closed position, according to some implementations of the present disclosure;

FIG. 14C is a front perspective view of the apparatus of FIG. 12 in a closed position, according to some implementations of the present disclosure;

FIG. 14D is a rear perspective view of the apparatus of FIG. 12 in a closed position, according to some implementations of the present disclosure;

FIG. 15A is a cross-sectional view of a container containing a volatile substance and a side view of the apparatus of FIG. 12, according to some implementations of the present disclosure;

FIG. 15B is a cross-sectional view of the container of FIG. 14A with a portion of the apparatus inserted into an opening, according to some implementations of the present disclosure;

FIG. 15C is a cross-sectional view of the container of FIG. 14A with the apparatus fully inserted into an opening, according to some implementations of the present disclosure;

FIG. 15D illustrate the capillary action by which the volatile substance in the container is drawn up into the apparatus so that volatile substance is evaporated into the surrounding atmosphere, according to some implementations of the present disclosure;

FIG. 16A is a perspective view of a base for a container, according to some implementations of the present disclosure;

FIG. 16B is a plan view of the base of FIG. 16A, according to some implementations of the present disclosure;

FIG. 16C is a partial cross-sectional view of the base of FIG. 16A, a container, and the apparatus of FIG. 12, according to some implementations of the present disclosure; and

FIG. 17 is a plot illustrating an amount of oil-based fragrance emitted from apparatus that are the same as the apparatus of FIG. 12, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

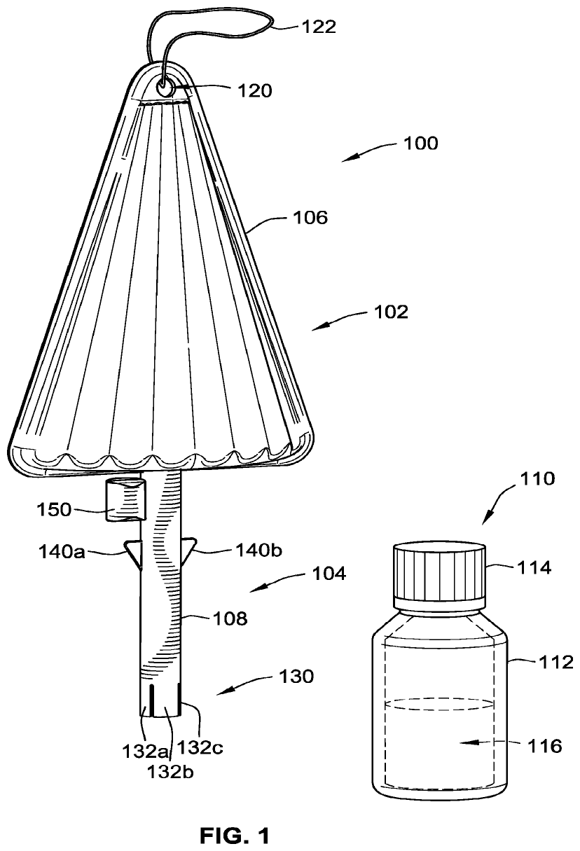

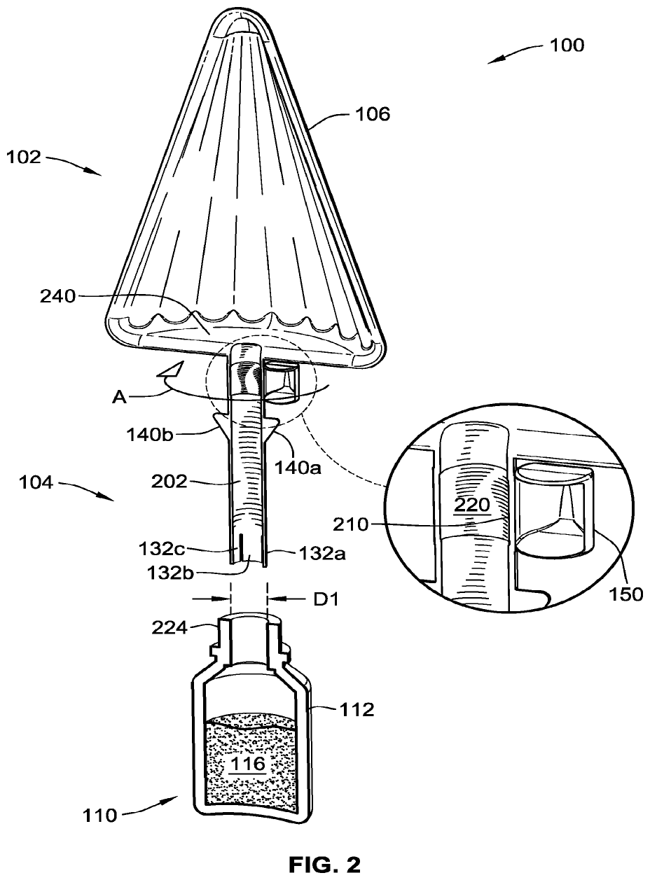

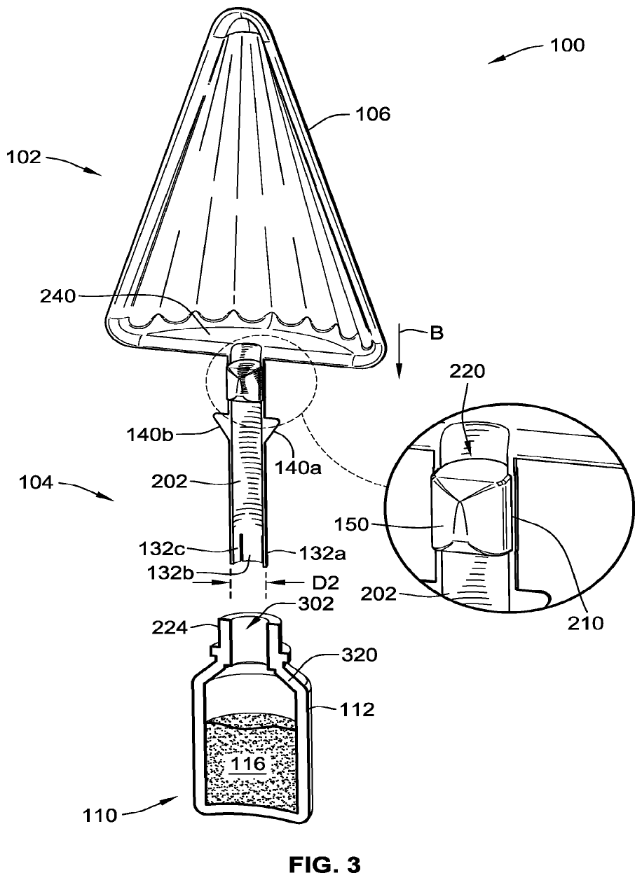

FIG. 1 illustrates an apparatus 100 for diffusing a volatile substance 116 to an ambient environment. The apparatus 100 includes an elongated wick portion 104 having a free end 130 configured to be inserted into a container 110 containing a volatile substance 116 that is drawn through capillary action into the elongated wick portion 104. The apparatus 100 includes an emanating portion 102 having an exposed surface 106 from which the volatile substance 116 evaporates into the ambient environment. The apparatus 100 includes a first protruding member 140a and a second protruding member 140b on the elongated wick portion 104, each member being configured to couple the apparatus 100 to the container 110 containing the volatile substance 116 without the apparatus 100 and container 110 uncoupling under a weight of the container 110. The wick portion 104 includes a plug portion 150 having a hinge member 210 (FIG. 2) coupled to the elongated wick portion 104. The plug portion 150 is configured to rotate about the hinge member 210 to form, together with the elongated wick portion 104, a plug 150 that is configured to be inserted into an opening 302 (FIG. 3) of the container 110 to suppress release of the volatile substance 116 due to evaporation via the opening 302 and to maximize dissemination of the volatile substance 116 through capillary action from the elongated wick portion 104 to the emanating portion 102.

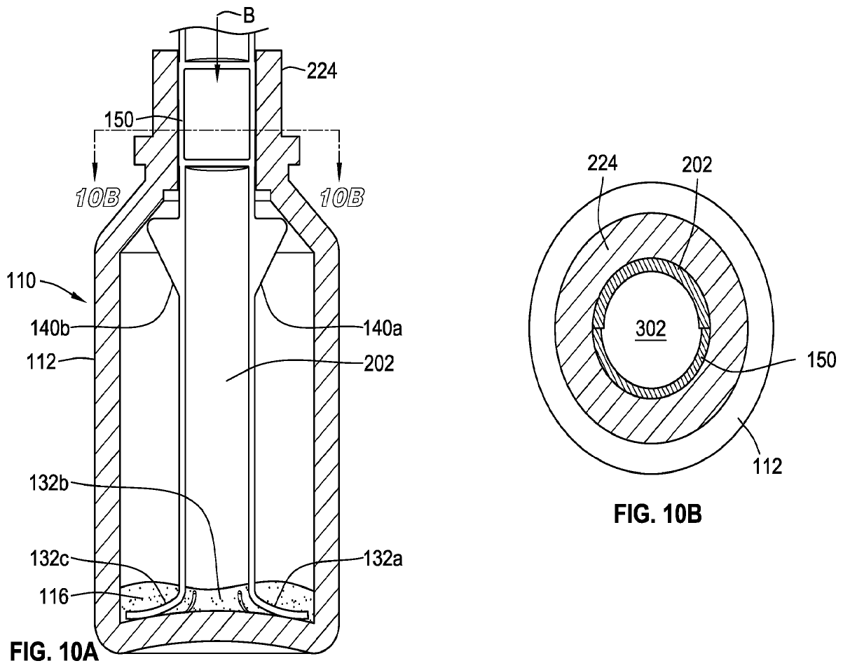

The hinge member 210 is a thinner thickness relative to the body 108 of the wick portion 104 to allow it to be flexible. The plug portion 150 resembles half of a soda can in shape so that when it is rotated in the direction of arrow A shown in FIG. 2 from its initial open position (FIG. 2) to a closed position (FIG. 3), it forms a plug once inserted into the neck 224 of the container 110. The neck 224 has an inner diameter, D1 (FIG. 2), and the body 202 of the wick portion 104 has an outer width, D2 (FIG. 3), which is substantially equal to D1 to ensure a snug fit. The diameter of the plug portion 150 (see FIG. 10B), is at least equal to D1 and can be slightly larger than D1 to ensure a snug, interference fit, when inserted through the neck 224 of the container 110. This snug fit ensures that none of the volatile substance 116 will spill out in case of accidental rotation of the container 110 toward a horizontal orientation, and will also minimize or suppress evaporation of the volatile substance 116 into the surrounding environment, ensuring that all or nearly all of the volatile substance 116 contained within the body 112 of the container 110 will be drawn up by capillary action out of the container 110 into the emanating portion 102 before being evaporated into the surrounding environment.

To aid in a snug fit, shown in FIG. 2, a recess 220 can be formed in the area of the wick portion 104 where the plug portion 150 is to be received against the body 202 of the wick portion 104. The enlarged section of FIG. 2 shows the plug portion 150 in an open position, which in this example has a soda can shape with a circular lid and bottom and a semi-cylindrical body. When rotated about the hinge 210, the lid and bottom of the plug portion 150 are received in the recess 220 to take into account the thickness of the plug portion 150 (see FIG. 3). The wick portion 104 is inserted through opening 302 into the neck portion 224 of the container 110 in the direction of arrow B (FIG. 3) to initiate the capillary action as described below.

The apparatus 100 constitutes a passive diffuser, meaning that no electric assistance by heat or active venting, e.g., electrically powered fan, is needed to disseminate the volatile substance into the surrounding atmosphere. The apparatus 100 is intended to be stationary (meaning it stays put against gravity) and has no actively moving parts, and can be hung like an ornament and would sway only from air movement in surrounding environment. For example, the apparatus 100 can be inserted into a container, which in turn is inserted into a stationary base.

The emitting portion 102 contributes significantly more to the regulation of the rate of release or evaporation of molecules of the volatile substance into the atmosphere than the wick portion 104.

The volatile substance 116 can be an active volatile liquid or gel, dispensing or diffusing by evaporation into surrounding environment. The liquid is at least partially volatile, and can evaporate under atmospheric pressure and normal ambient room temperatures, typically between 15-35° C.

An object of the apparatus 100 is to control the rate of release or evaporation of a volatile substance into the surrounding environment. As will be discussed herein, this control comes in the form of suppressing evaporation or possibility of spillage from the container 110, the shape and geometry of the emanating portion 102, the length and cross-sectional area of the wick portion 104, and so forth. The primary action that causes the volatile substance 116 to be diffused into the atmosphere is capillary action in which the volatile substance 116 is drawn into the wick portion 104 and then into the emanating portion 102 where it is released by evaporation into the surrounding environment.

The volatile substance 116 can include a perfume, fragrance, odor, scent, fragranced odor, odor suppressing or masking agents, insect-repellent, air freshener, deodorant, odorant, germicide, larvicide, disinfectant, insecticide, rodent repellent, or bactericide. The volatile substance 116 can include natural oils or extracts such as pine, mint, lavender, cinnamon, cedar, lemon, and other essential oils and extracts. A dye can be added to the volatile substance 116 to cause the apparatus 100 to change color as the dyed volatile substance diffuses throughout the surface of the apparatus by capillary action. In a specific aspect, an oil-based color dye can be introduced to an oil or oil extract to produce a colored oil that, when by capillary action, the dyed oil permeates to its visible surface, the color of apparatus 100 changes to correspond to the color of the dye (accounting for the starting color of the apparatus 100, which may result in a slightly darker shade of the color perceived by the naked eye). A “fragrance” as used herein does not necessarily have to be pleasing, e.g., to humans, but can be displeasing, e.g., to predators, pests, or the like.

The wick portion 104 and the emanating portion 102 can be composed of a wick-absorbent material, cardboard, sugarcane, sugarcane bagasse, plant fiber, cellulose, cotton, linter-based material fibers, cellulose derivatives, papers, molded ceramics, sintered or porous plastics, organic and inorganic materials, wood flour, felt, cotton, tissue, pulp, woven and non-woven cotton fibers, synthetic fibers, cellulose derivatives, nylon, open cell, or polyethylene. The wick portion 104 and the emanating portion 102 can be made of the same material and form a unitary piece, such as formed from a single mold. The wick portion 104 and the emanating portion 102 can be made from thermoformed molded pulp (Type 3) (wet press or dry press) or from transfer molded fiber (Type 2). In one example, the wick portion 104 and emanating portion 102 comprise thermoformed molded sugarcane bagasse (Type 3). The sugarcane bagasse can be white, black or natural (e.g., no color added). The apparatus 100 can be generally rigid (maintain its shape without sagging or curling over time), or the apparatus 100 can be non-rigid or semi-flexible, such as the protruding members 140a, 140b, which can be compressed or squeezed against the body of the wick portion 104 and then returned back to their original position.

The designations “emanating,” “emitting,” “releasing,” “dispersing,” “expressing,” and “diffusing,” all applying to the emanating portion, part or piece of the structure from which the volatile substance typically evaporates into the surroundings, shall be used herein interchangeably. The emanating portion 102 includes an evaporative surface or area 106 and can assume any shape or geometry.

By “wick” it is understood here essentially the part of the wick/emitting structure of the apparatus 100, which is plunged in the volatile substance 116 contained in the reservoir 110, once the apparatus 100 is activated.

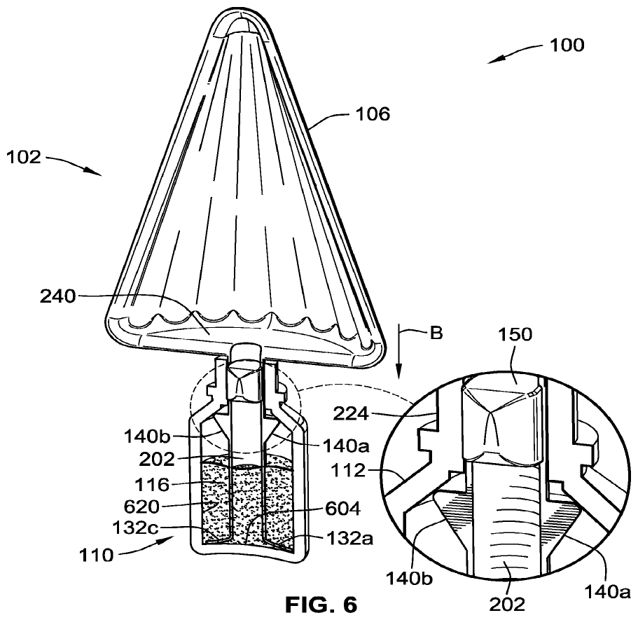

Returning to the figures, the container 110 can have a bottle shape and a screw cap 114 over the opening 302 and a shoulder 430 between the opening 302 and a body 112 of the bottle 110. The volatile substance 116 can include a fragrance oil, and the combination of the apparatus 100 and the container 110 (when filled with the volatile substance 116) can have a weight that does not exceed two ounces. The protruding members 140a, 140b are configured to be inserted into the opening 302 and past the shoulder 320 until the members 140a, 14b rest against the shoulder 320 inside the container 110 (see FIG. 6).

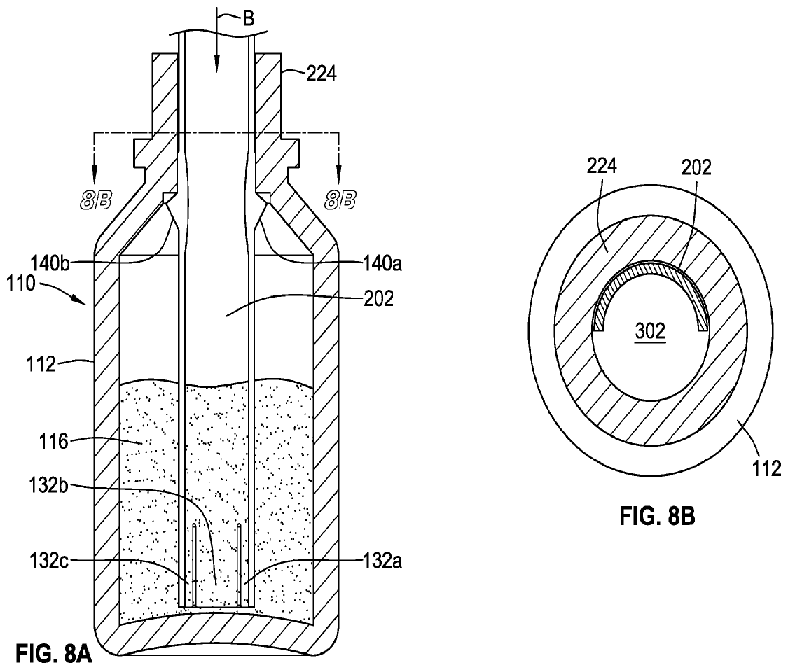

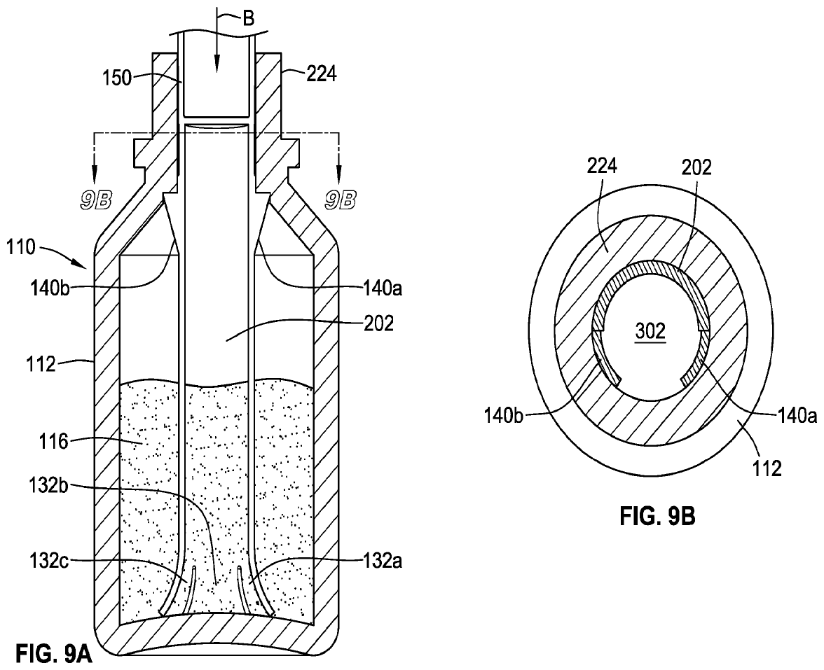

The elongated wick portion 104 has a semi-circular cross-section 202 (FIG. 8B), and the plug portion 150 has a semi-circular cross-section (FIG. 10B) such that when the plug portion 150 is joined with the elongated wick portion 104, the plug 150 has a generally circular cross-section that substantially closes the opening 302 to the container 110 (compare FIG. 9B with FIG. 10B).

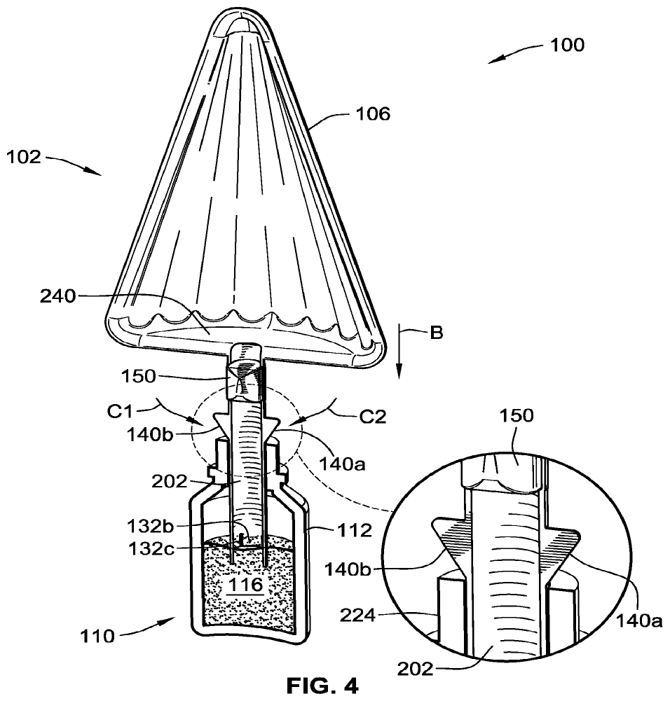

The first protruding member 140a and the second protruding member 140b protrude away from the elongated wick portion 202 (FIG. 3) and have a tapered portion (enlarged in FIG. 4) that tapers toward the elongated wick portion 202 toward the free end 130.

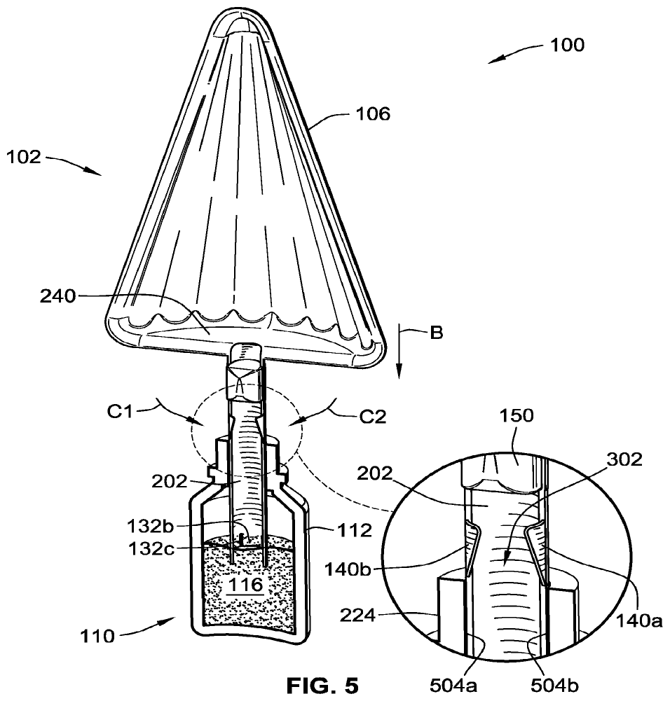

The first protruding member 140a and the second protruding member 140b is flexible to squeeze into the opening 302 of the container 110 until they expand once moved beyond the opening 302 of the container 110 (see FIGS. 5-6 and 8A, 8B, 9A, 9B, 10A, and 10B). The first and second protruding members 140a,b can rotate or flex in the directions of arrows C1, C2, respectively, shown in FIG. 4, for example, to squeeze into the neck 224 of the container 110 as the wick portion 104 is inserted through the opening 302 in the direction of arrow B shown in FIG. 4. Then, as the wick portion 104 continues to move toward the bottom of the container 110, the first and second protruding members 140a,b begin to deform, flex, or fold as shown in FIG. 5 in the direction of arrows C1, C2, respectively, about the body 202 of the wick portion 104. Some slight deformation of the sidewalls of the body 202 of the wick portion 104 may occur, as shown in FIG. 8A, until the first and second protruding members 140a,b clear the shoulder 320 just after the neck 224 of the container 110.

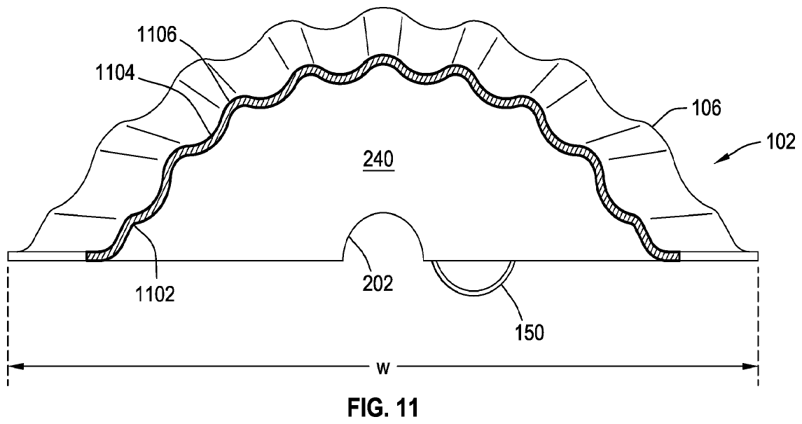

The free end 130 of the elongated wick portion 104 has flexible legs 132a,b,c, (three in this example) each of which is configured to bend and extend toward sidewalls 620 (FIG. 6) of an interior of the body 112 of the container 110 in response to the elongated wick portion 104 being inserted through the opening 302 until the free end 130 contacts a bottom interior surface 604 (FIG. 6) of the container 110, and each of the flexible legs 132a,b,c splay out away from one another. Although three legs are shown, two or more than three are contemplated. The use of the term leg is not intended to convey any particular shape. The legs are extensions of the wick portion 104 that flex, bend, fold, or otherwise deform to reach otherwise inaccessible areas inside the container 110 to draw up any remaining pooling volatile substance as it is nearly used up over time. This overcomes a problem with conventional reed diffusers, which require the reeds to be manually moved around (e.g., revered 180 degrees) to drink up any residual oil remaining in the bottom of the diffuser container, assuming this movement occurs before evaporation claims the rest of the residual oil. Here, the plug portion 150 eliminates or suppresses the chance for any evaporation to occur from the now-plugged opening 302 of the container 110, allowing all or nearly all of the volatile substance 116 to be used up by capillary action by traversing into the wick portion 104 and then into the emanating portion 102 before being evaporated into molecules into the surrounding atmosphere external to the emanating portion 102. When the volatile substance is oil-based, it has been found that the emanating portion 102, having approximate dimensions of 3 inches in width (dimension W shown in FIG. 11) and about 4.6 inches in height (along the vertical direction in the orientation shown in FIG. 1), and when the emanating portion 102 is made from thermoformed molded pulp (Type 3), when fully soaked in oil, the emanating portion 102 retains its structural integrity and does not get soggy and is not prone to disintegration over a period of time that would far exceed the time to consume all of the volatile substance 116 in the container 110. The wick portion 104 in the illustrated examples has a length (along its elongated dimension) of about 2 inches. Thus, when the entire apparatus 100 and container 110 are hung, the hole 120 remains intact and will not rupture the emanating part 102, so there is no risk that the ornament would fall even if left unattended for many weeks.

Returning to FIG. 6, the flexible legs 132a,b,c can have any number and take any shape. The idea here is to have a part of the wick portion 104 that can cover some or all of the bottom 604 of the container 100 to ensure all of the available volatile substance 116 is drawn up the wick portion 104 by capillary action. For example, the legs 132 can take the form of a rake or mop so that when the wick portion 104 reaches the bottom 604 and is pressed into the bottom 604, the legs can spread out like fingers or like a mop to soak up the volatile substance 116. This configuration is particularly useful when the bottom surface 604 of the container 110, such as shown in FIG. 6, is slightly convex (relative to the exterior bottom of the container 110) and not completely flat. The convex shape of the bottom surface 604 actually aids in spreading the legs 132 apart in the directions away from each other, so this feature can actually be exploited by the apparatus 100 during assembly.

The apparatus 100 can include a hole 120 (FIG. 1) in the emanating portion 102 to receive therethrough a hanging element 122 (e.g., a hook or a string) for hanging the apparatus 100 as an ornament, e.g., to an artificial or real tree branch. Alternately, the apparatus 100 can be free-standing (e.g., resting on a surface). Advantageously, the contents of the container 110 can be refilled once the volatile substance 116 has been depleted, allowing the capillary action to resume. As discussed above, when the volatile substance is oil as opposed to water-based, and the apparatus 100 is composed of a thermoformed molded pulp, the structural integrity is not compromised once soaked in oil, allowing many repeated refills of the container without having to discard the apparatus 100.

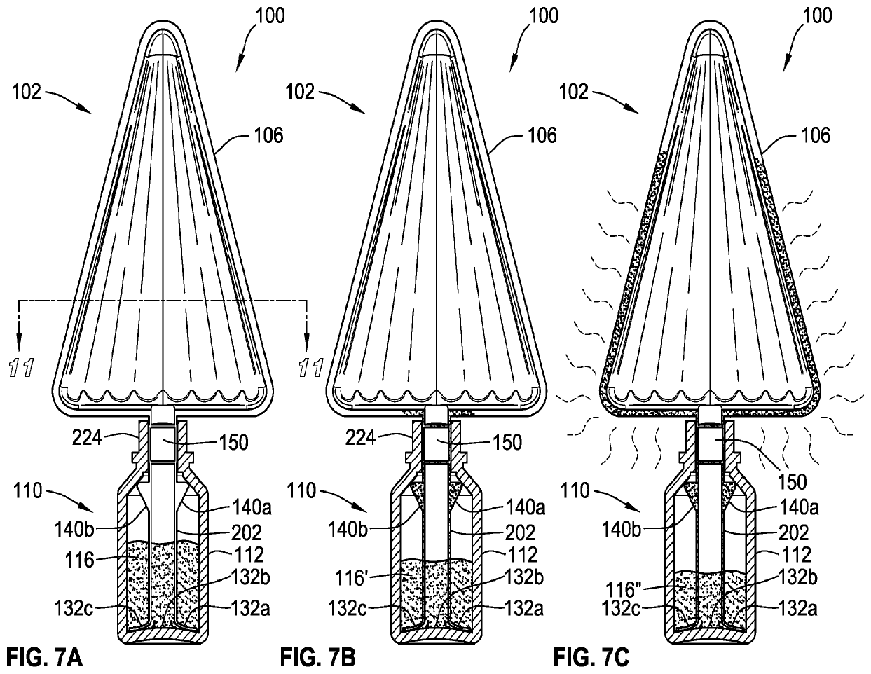

The emanating portion 102 has a dimension across its surface 106 along a cross-section of the emanating portion 102. This dimension is at least 10% or at least 20% or at least 30% or at least 40% or at least 50% greater than a width, W, of the emanating portion 102 along the same cross-section, which is best seen in FIG. 11. The surface 106 of the emanating portion 102 includes a wavy cross-section with peaks and valleys 1104, 1106 to increase its overall surface area. The overall dimension of the cross-section 1102 taken from the cross-section shown in FIG. 7A and illustrated in FIG. 11 can be seen to be greater (about 10%) than a width, W, of the emanating portion 102. An example width of the emanating portion is 3 inches. Instead of a wavy shaped, the surface 106 can include saw-tooth structures, like a lamp shade, to increase surface area.

The elongated wick portion 104 and the emanating portion 102 can form a unitary, integral piece composed of a thermoform or thermoformed molded pulp. Alternately, the elongated wick portion 104, the emanating portion 102, the protruding members 140a, b, and the plug portion 150 are formed as a unitary, integral piece from a thermoform molded pulp introduce to a mold. The thermoformed molded pulp can be of Type 3, which is also referred to as a “cure in the mold” type, where the wet/damp pulp is poured into the mold and cured in situ as the moisture is removed using heat.

FIGS. 7A, 7B, and 7C illustrate the capillary action of the volatile substance 116 once the wick portion 104 has been inserted into the container 110 filled with the volatile substance 116. The relative levels of the remaining volatile substance 116 have been exaggerated for ease of illustration and discussion. In reality, the levels would imperceptibly lowered during the initial uptake of volatile substance 116. As soon as the wick portion 104 makes contact with the volatile substance 116, capillary action will begin to draw up the volatile substance 116 into the wick portion 104 as can be seen in FIG. 7B while drawing down (slightly) the quantity of available volatile substance 116. Because the wick portion 104 is uninterrupted from the legs 132 to the emanating portion 102, opportunities exist for the volatile substance 116 to traverse even against gravity to find accommodating places to travel. Note that the skilled person will appreciate that the pressure inside the container 110 and outside the container should be approximately the same to permit capillary action to occur, or at least the pressure inside the container should not be lower than atmospheric pressure of the surrounding environment. Under these conditions, the volatile substance 116 will eventually soak into the distal most (relative to the free end 130) portions of the apparatus 100, as can be seen in FIG. 7C. The wavy lines convey an emission of fragrance or other molecules once the volatile substance 116 is converted into an evaporated gas form into the surrounding environment.

Another way of describing the coupling between the apparatus 100 and the container 110 is by the number of points of contact. In one example, there are at least four points of contact to hold and support the container 100 while the entire assembly (container+apparatus) is hung as an ornament. Those four points of contact between the apparatus 100 and the container 110 include: the plug 150, the protruding members 140a, 140b, and the wick portion 104 itself. The amount of volatile substance in the container 100 can be about 0.5 ounces, or 15 grams.

While the emanating portion 102 has been shown in this disclosure as resembling or having a tree shape, the shape or form factor of the emanating part 102 is not a salient aspect of the present disclosure. Any other shape or form factor can be envisioned. For example, the emanating part 102 can be have a candy cane shape, and the volatile substance can include a peppermint oil. The emanating 102 can resemble a cinnamon stick with the volatile substance including cinnamon oil.

To assemble the apparatus 100 into a fragrance-emitting ornament or freestanding device, the user rotates the plug portion 150 to close it and form a plug. The screw cap 114 is unscrewed from the container 110, and the free end 130 of the wick portion 104 is inserted through the opening 302 of the container into the neck 224 of the container 110. A slightly resistance can be felt once the protruding members 140a,b hit the opening, but the user simply applies slightly more downward force to force the members 140a,b to squeeze through the neck 224 until they are clear of the shoulder 320 of the container 110. At the same time, the legs 132, thanks to the downward pressure, simultaneously splay out once they hit the bottom 604 of the container 110 until the user can no longer press the elongated wick portion 104 in a downward direction (B) any further. Optionally, a hook or string 122 is passed through the hole 120 in the emanating part 102, and the entire apparatus 100 together with the container 110 attached can be hung, e.g., from a tree branch.

When the volatile substance 116 is an oil-based fragrance and the apparatus 100 is molded from Type 3 thermoformed molded pulp, using the example dimensions provided herein, a large room can be permeated with a pleasing fragrance, such as, for example, a 144 square foot room with an 8 foot ceiling. When used for Christmas holiday, for example, the volatile substance 116 can last the entire holiday season, and can even be refilled to extend its fragrance-emitting life. To stop or pause fragrance emission, the user can remove and discard or hermitically store the wick portion 104 from the container 110 and screw the cap 114 back onto the container 110. By “oil-based,” it is meant that the volatile substance includes oil such as 100% oil that can include a fragrance, which may be pleasing (e.g., to humans) or not pleasing (e.g., to pests or predators), or a perfume, odor, scent, fragranced odor, odor suppressing or masking agent, insect-repellent, air freshener, deodorant, odorant, germicide, larvicide, disinfectant, insecticide, rodent repellent, bactericide, or any combination of the foregoing.

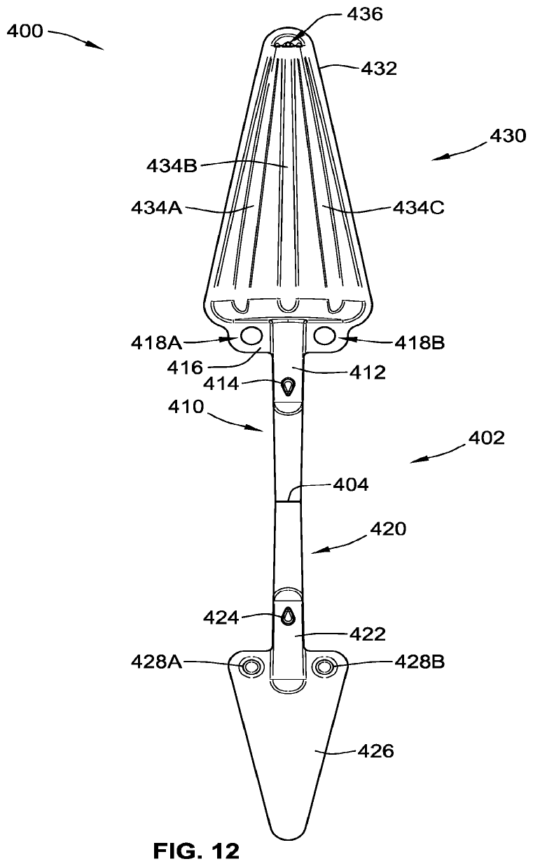

Referring to FIG. 12, an apparatus 400 that is similar to the apparatus 100 described herein is shown in that apparatus 400 can be used for diffusing a volatile substance (e.g., an oil-based fragrance) to an ambient environment.

The apparatus 400 includes a wick portion 410 and an emanating portion 430. The wick portion 410 and the emanating portion 430 can be composed of a wick-absorbent material, cardboard, sugarcane, sugarcane bagasse, plant fiber, cellulose, cotton, linter-based material fibers, cellulose derivatives, papers, molded ceramics, sintered or porous plastics, organic and inorganic materials, wood flour, felt, cotton, tissue, pulp, woven and non-woven cotton fibers, synthetic fibers, cellulose derivatives, nylon, open cell, or polyethylene. For example, in some implementations, the wick portion 410 and the emanating portion 430 comprise a thermoform molded molded sugarcane bagasse (Type 3). The sugarcane bagasse can be white, black or natural (e.g., no color added). The thermoformed molded pulp can be of Type 3, which is also referred to as a “cure in the mold” type, where the wet/damp pulp is poured into a mold and cured in situ as moisture is removed using heat. Alternatively, the wick portion 410 and/or emanating portion 430 can comprise transfer molded fiber (Type 2).

The wick portion 410 and the emanating portion 430 can be made of the same material and form a unitary piece. For example, the wick portion 410 and the emanating portion 430 can be formed together in a single mold. Alternatively, in some implementations, the wick portion 410 and the emanating portion 430 can be formed as separate and distinct components composed of the same or different material and coupled together (e.g., adhered together).

The wick portion 410 includes a first portion 410 and a second portion 420 that are configured to be at least partially inserted into an opening of a container (e.g., the container 110 described herein). The wick portion 410 includes a hinge 404 positioned between the first portion 410 and the second portion 420 to permit the first portion 410 to move relative to the second portion 420 (or vice versa). Relative movement of the first portion 410 and second portion 420 permits the apparatus 400 can move from the open position shown in FIG. 12 to a closed position, as described further herein. The hinge 404 can be formed as a scored or perforated line to aid in permitting of the first plug portion 410 and second plug portion 420 relative to one another.

The first portion 410 includes a first plug portion 412, a first sealing protrusion 414, a first tongue 416, and a pair of apertures 418A-418B. The first plug portion 412 is configured to be at least partially inserted into an opening of a container (e.g., the opening 302 of the container 110 described herein). The first plug portion 412 is generally semi-cylindrical shaped so as to form a cylindrical plug with a portion of the second plug portion 420, as described further below. The first sealing protrusion 414 has a generally tear-drop shape and is configured to engage a portion of the container to aid in securing the apparatus 400 to the container, as described in further detail below. The tongue 416 is generally positioned between the first plug portion 412 and the emanating portion 430.

The pair of apertures 418A-418B are formed in the tongue 416 and can have a diameter of, for example, about 4 mm. As described below, the pair of apertures 418A-418B engage a portion of the second portion 420 to aid in coupling the first portion 410 to the second portion 420. The pair of apertures 418A-418B can be formed as part of the mold used to form the apparatus 400 or punched out after forming the first portion 410 in the mold.

The second portion 420 includes a second plug portion 422, a second sealing protrusion 424, a second tongue 426, and a pair of protrusions 428A-428B. The second plug portion 422 is configured to be at least partially inserted into an opening of a container (e.g., the opening 302 of the container 110 described herein). The second plug portion 422 is generally semi-cylindrical shaped so as to form a cylindrical plug when positioned adjacent to the first plug portion 412. The second sealing protrusion 424 is the same as, or similar to, the first sealing protrusion 414.

The tongue 426 has a generally triangular shape. As described in further detail herein, when the first plug portion 412 and second plug portion 422 are inserted into an opening of a container, the tongue 426 is not positioned within the container such that the tongue 426 provides additional surface area for emanating a substance from the container that is drawn into the apparatus via capillary action. The pair of protrusions 428A-428B have a generally circular or dome shape and are formed on second tongue 426. The pair of protrusions 428A-428B are configured to engage corresponding ones of the pair of apertures 418A-418B to aid in coupling the first plug portion 410 to the second plug portion 420, as described further herein.

At least a portion of the wick portion 402 of the apparatus 400 can be inserted within an opening of a container (e.g., the container 110) to draw a substance (e.g., the substance 116) into the apparatus 400 via capillary action. The emanating portion 430 includes an evaporative surface or area 432 from which the volatile substance drawn up into the emanating portion 430 via the wick portion 402 evaporates into the ambient environment. The emanating portion 430 also includes a hole 436 to receive therethrough a hanging element (e.g., a hook or a string) for hanging the apparatus 400 as an ornament, e.g., to an artificial or real tree branch. Alternatively, the apparatus 400 can be free-standing (e.g., resting on a surface), as described below.

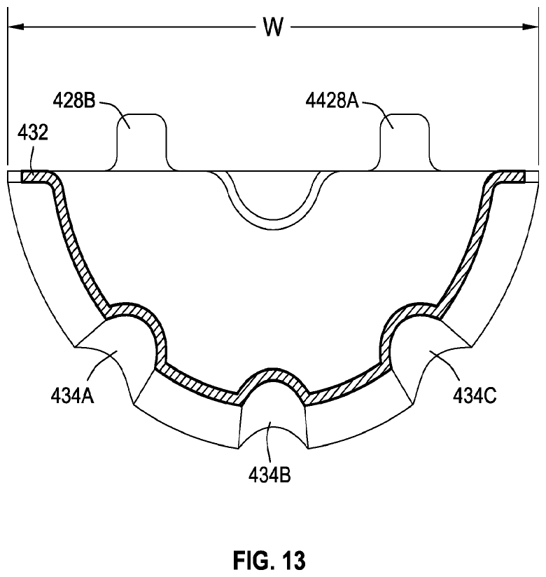

As shown in FIG. 12 and the cross-sectional view of FIG. 13, evaporative surface or area 432 of the emanating portion 430 has a generally curved cross-sectional profile and includes a plurality of grooves 434A-434C that increase the overall surface area of the evaporative surface or area 432. While shown as including three, grooves 434A-434C, the evaporative surface or area 432 can include any suitable number of grooves for increasing the surface area of the evaporative surface or area 432 (e.g., 2, 4, 8, 10, etc.). As shown in FIG. 12, the emanating portion 430 has a generally triangular or tapered shape (e.g., a shape that resembles a tree) where a first width of the emanating portion 430 adjacent to the first plug portion 410 of the wick portion 402 is greater than a second width of the emanating portion 430 at its distal end (e.g., opposite the wick portion 402 and adjacent to the hole 436). The first width of the emanating portion 430 can be between about 1.1 and about 10 times greater than the second width, between about 2 and about 6 times greater, between about 3 and about 5 times greater. For example, the first width can be approximately 2.25 inches and the second width can be approximately 0.5 inches (e.g., the first width is about 4.5 times greater than the second width). As described further below, at least the first width is greater than a diameter of the opening of the container in which the wick portion 402 is inserted (e.g., between about 2 times and about 6 times greater than the diameter of the opening). The emanating portion 430 also has a length that is greater than both the first width and the second width (e.g., about 2 times greater than the first width). For example, the length of the emanating portion 430 (e.g., measured from the top of the first tongue 416 to the distal end adjacent the hole 438) that is between about 3 inches and about 6 inches (e.g., about 4.5 inches). In some implementations, the thickness of the emanating portion 430 is about 0.032 inches.

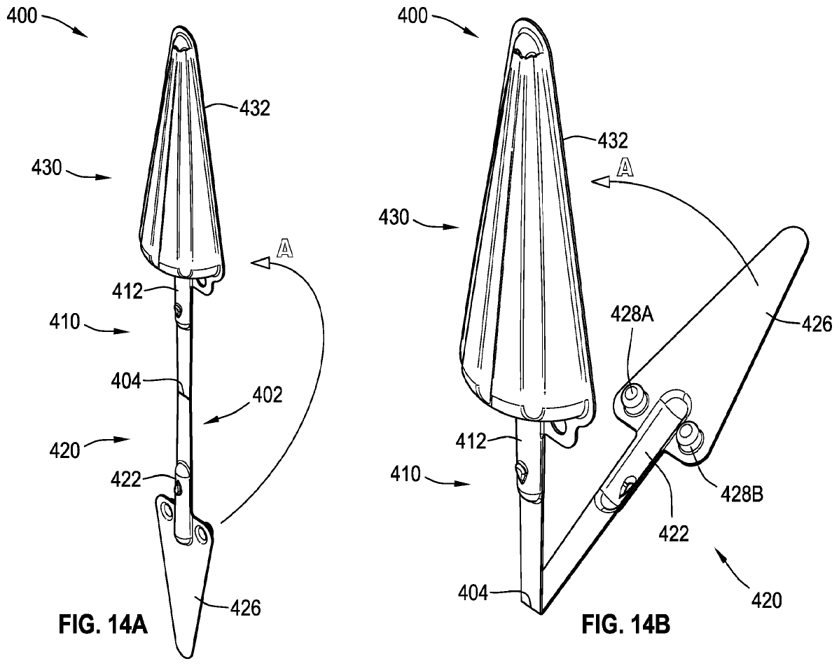

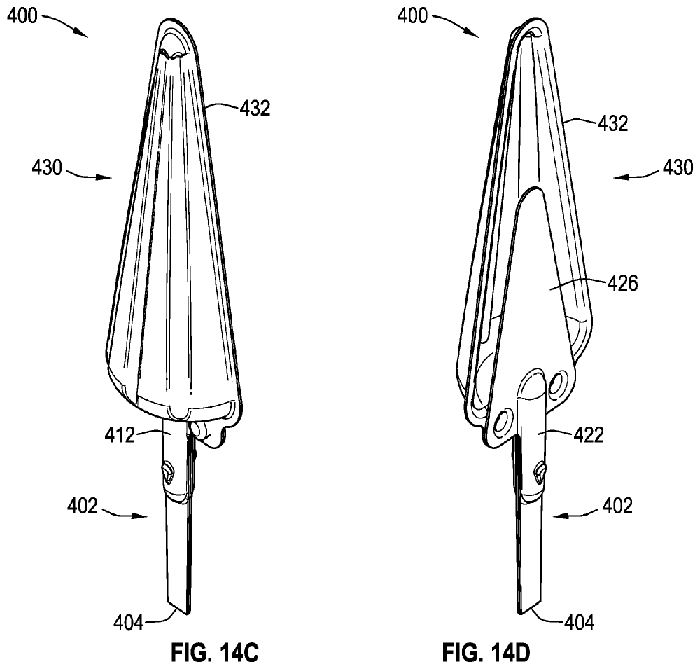

In FIG. 12, the apparatus 400 is illustrated in a first or open position where the hinge 404 is not bent. Referring to FIGS. 14A-D, the apparatus 400 can be transition from an open position (FIG. 14A) to a closed position (FIGS. 14C-D) so that the wick portion 402 of the apparatus 400 can be inserted into a container containing a substance (e.g., the container 110 described herein). In FIG. 14A, like FIG. 12, the apparatus 400 is in an open position. To transition the apparatus 400 towards a closed position, the second portion 420 can be moved relative to the first portion 410 via the hinge 404 by moving the second plug portion 420 in the direction of arrow A, as shown in FIG. 14B. For example, a user can hold the emanating portion 430 and/or first portion 410 and move the second portion 420 in the direction of arrow A. The user can continue moving the second portion 420 in the direction of arrow A until the pair of protrusions 428A-428B on the second tongue 426 engage the pair of apertures 418A-418B (FIG. 12) of the first tongue 416, as shown in FIGS. 14C-D. The pair of protrusions 428A-428B form a press or interference fit with the pair of apertures 418A-418B to couple the first plug portion 410 and the second portion 420 and to aid in inhibiting relative movement of the first portion 410 and the second portion 420. In the closed position, the first plug portion 412 and the second plug portion 422 form a generally cylindrical plug that can be inserted into the opening of a container.

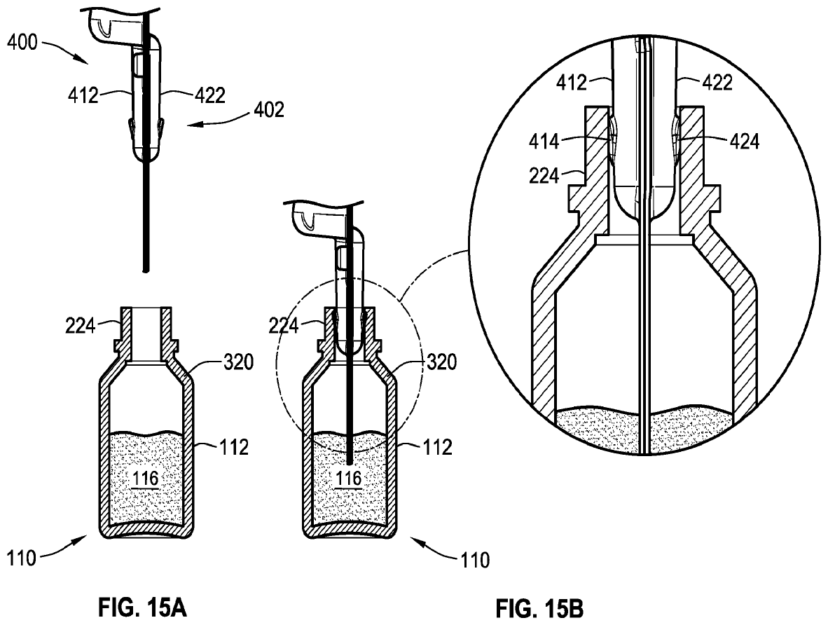

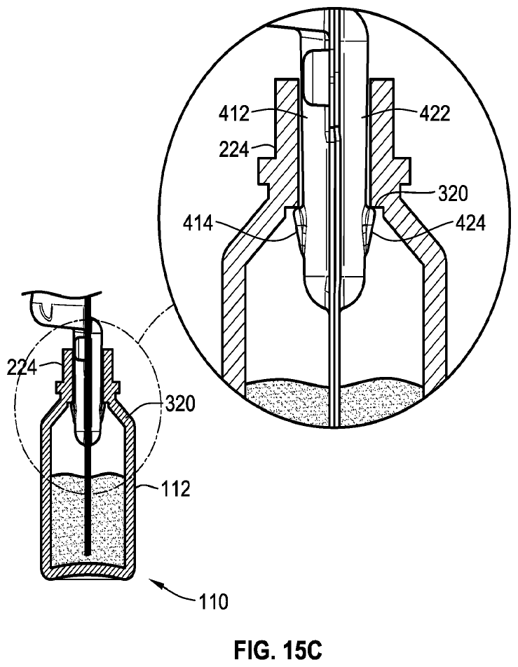

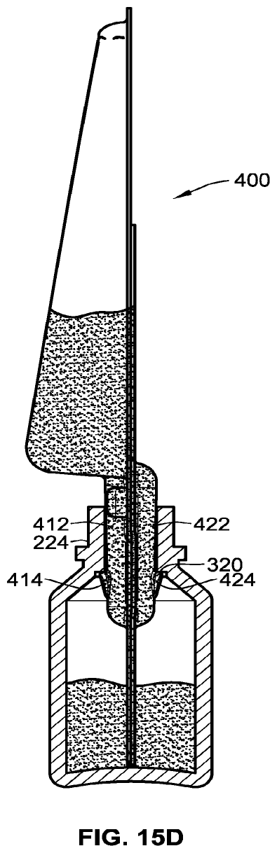

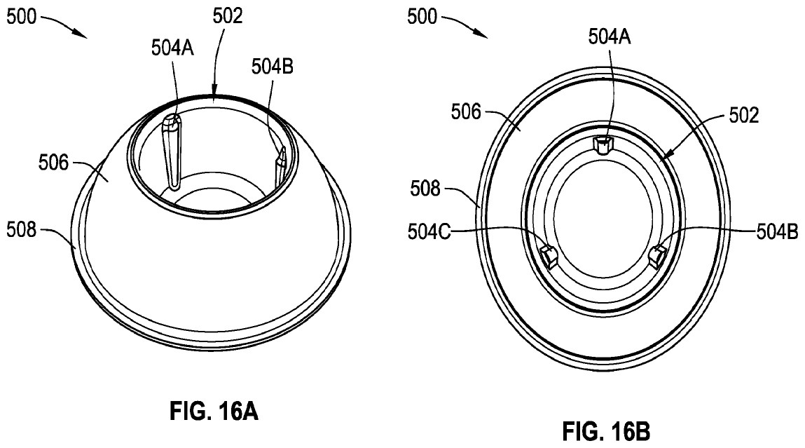

The apparatus 400 can be inserted into the container 110 described herein to diffuse the substance 116 stored therein to an ambient environment. FIGS. 15A-D illustrate the apparatus 400 being inserted into the opening 302 of the container 110 and the capillary action of the volatile substance 116 once the wick portion 402 has been inserted into the container 110 filled with the volatile substance 116. The relative levels of the remaining volatile substance 116 have been exaggerated for ease of illustration and discussion. In reality, the levels would imperceptibly lowered during the initial uptake of volatile substance 116.

In FIG. 15A, the apparatus 400 is moved to the closed position described above (FIGS. 14C-D) and the wick portion 402 is positioned generally adjacent to the opening 302 of the container 110. As the apparatus 400 is moved in the direction of arrow B towards the container 110, the hinge 404 enters the opening 302 of the container 110 first. Then, the first sealing protrusion 414 and the second sealing protrusion 424 are deflected or deformed (e.g., elastically deformed) as the first plug portion 412 and second plug portion 422 move into the opening 302 of the container 110, as shown in FIG. 15B.

As the apparatus 400 continues to move in the direction of arrow B, the first sealing protrusion 414 and the second sealing protrusion 424 eventually are no longer deflected or deformed by the neck 224 and engage the shoulder 320 of the container 110, as shown in FIG. 15C. The first sealing protrusion 414 and the second sealing protrusion 424 aid in securing the apparatus 400 to the container 110 (e.g., such that if a user picked up the apparatus 400 from the emanating portion 430, the container 110 would be picked up along with the apparatus 400). The first sealing protrusion 414 and the second sealing protrusion 424, along with the first plug portion 412 and the second plug portion 422 also form a substantially air-tight seal that inhibits the substance 116 from escaping the container 110 either due to evaporation or the container 110 tipping over.

As soon as the wick portion 402 makes contact with the volatile substance 116, capillary action will begin to draw up the volatile substance 116 into the wick portion 402 as can be seen in FIG. FIG. 15 while drawing down (slightly) the quantity of available volatile substance 116. Because the wick portion 402 is uninterrupted from the hinge 404 to the emanating portion 430, opportunities exist for the volatile substance 116 to traverse even against gravity to find accommodating places to travel. Note that the skilled person will appreciate that the pressure inside the container 110 and outside the container should be approximately the same to permit capillary action to occur, or at least the pressure inside the container should not be lower than atmospheric pressure of the surrounding environment. Under these conditions, the volatile substance 116 will eventually soak into the distal most (relative to the hinge 404) portions of the apparatus 400, as can be seen in FIG. 15D. The wavy lines convey an emission of fragrance or other molecules once the volatile substance 116 is converted into an evaporated gas form into the surrounding environment.

When the volatile substance 116 is an oil-based fragrance and the apparatus 400 is molded from Type 3 thermoformed molded pulp, using the example dimensions provided herein, a large room can be permeated with a pleasing fragrance, such as a 500, 750, or 1000 square foot room having a ceiling height of 10 feet. When used for the Christmas holiday, for example, the apparatus 400 can diffuse fragrance from the entire holiday season (e.g., about 1 month), and the container can even be refilled to extend its fragrance-emitting life.

In some implementations, the apparatus 400 diffuses the volatile substance drawn up via the wick portion 402 into the ambient environment at a predetermined average rate of release. Scented fragrance devices preferably diffuse enough fragrance so that it is perceptible and pleasing to users. However, as described above, many conventional diffusers suffer from diminishing rates of scent release over time. The apparatus 400 overcomes the drawbacks and limitations of conventional diffusers, among other ways, by having an average rate of release that is greater than about 0.25 grams of the volatile substance per day over 30 days.

In a first example, an apparatus comprising thermoform molded sugarcane pulp having the same shape as the apparatus 400 was tested to demonstrate the average rate of release of a 100% oil fragrance (pure oil) over 45 days. The emanating portion of the apparatus had a length of about 4.5 (measuring from the top of the first tongue to the distal end adjacent the hole), a width measured adjacent to the plug of about 2.25 inches, a width measured adjacent at the distal end of about 0.5 inches, and a thickness of about 0.032 inches. During the testing, the apparatus was inserted in the container during the entirety of the test and the ambient temperature of the environment into which the oil-based fragrance was released was maintained at approximately 72 degrees Fahrenheit. The rate of release was measured by weighing the apparatus and the container storing the oil-based fragrance at the beginning of the test when the apparatus was inserted into the container. In this example, the starting weight was 54.12 grams. As shown in Table 1 below, the weight was measured in daily intervals to show how much of the oil-based fragrance was diffused into the ambient environment (measured in grams).

TABLE 1

| day | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| output | .74 | .52 | .52 | 1.06 | .84 | .46 | .51 | .42 | .69 | .69 | .7 | .49 | .32 | .39 | .21 |
| day | 16 | 17 | 18 | 19 | 20 | 21 | 22 | 23 | 24 | 25 | 26 | 27 | 28 | 29 | 30 |
| output | .21 | .44 | .21 | .23 | .27 | .23 | .36 | .13 | .14 | .15 | .19 | .16 | .17 | .18 | .13 |
| day | 31 | 32 | 33 | 34 | 35 | 36 | 37 | 38 | 39 | 40 | 41 | 42 | 43 | 44 | 45 |
| output | .11 | .11 | .13 | .12. | .1 | .08 | .02 | .02 | .07 | .06 | .06 | .04 | .04 | .03 | .01 |

In this first test, approximately 0.11 grams of the oil-based fragrance was released in the environment 5 hours after inserting the apparatus into the container. Based on the starting weight and the daily output, the average rate of release over 45 days was approximately 0.28 grams/day. Over 30 days, the average rate of release was approximately 0.39 grams per day. Further, for each of the first 35 days, the apparatus released at least 0.1 grams of the volatile oil-based fragrance each day. During the first week (days 1-7), the apparatus released at least about 0.4 grams of the volatile oil-based fragrance each day and had an average rate of release of about 0.66 grams/day. During the second week (days 8-14), the apparatus released at least about 0.32 grams of the volatile oil-based fragrance each day and had an average rate of release of about 0.53 grams/day. During the third week (days 15-21), the apparatus released at least about 0.21 grams of the volatile oil-based fragrance each day and had an average rate of release of about 0.26 grams/day. During the fourth week (days 22-28), the apparatus released at least about 0.13 grams of the volatile oil-based fragrance each day and had an average rate of release of about 0.19 grams/day. During the fifth week, (days 29-35), the apparatus released at least about 0.1 grams each day and had an average rate of release of about 0.13 grams/day. During the sixth week (days 36-42), the apparatus released at least about 0.02 grams each day and had an average rate of release of about 0.05 grams/day.

In a second example, an apparatus that is the same as, or similar to, the apparatus used in the first example above was tested to demonstrate the average rate of release of the same oil-based fragrance over 45 days under the same conditions as in Table 1. In this example, the starting weight of the apparatus and container was 53.83 grams. As shown in Table 2 below, the weight was measured in daily intervals to show how much oil-based fragrance was diffused into the ambient environment (measured in grams).

TABLE 2

| day | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| output | .65 | .51 | .50 | 1.06 | .82 | .42 | .50 | .43 | .32 | .32 | .67 | .47 | .31 | .38 | .20 |
| day | 16 | 17 | 18 | 19 | 20 | 21 | 22 | 23 | 24 | 25 | 26 | 27 | 28 | 29 | 30 |
| output | .20 | .43 | .21 | .22 | .24 | .23 | .34 | .13 | .13 | .14 | .16 | .18 | .16 | .16 | .12 |
| day | 31 | 32 | 33 | 34 | 35 | 36 | 37 | 38 | 39 | 40 | 41 | 42 | 43 | 44 | 45 |
| output | .11 | .11 | .13 | .14 | .07 | .10 | .03 | .03 | .07 | .07 | .03 | .03 | .06 | .06 | .01 |

In this second test, approximately 0.08 grams of the oil-based fragrance was released in the environment 5 hours after inserting the apparatus into the container. Based on the starting weight and the daily output, the average rate of release over the 45 days was approximately 0.26 grams/day. Over 30 days, the average rate of release was approximately 0.35 grams per day. Further, over the first 34 days, the apparatus diffused at least 0.1 grams of the oil-based fragrance every day. During the first week (days 1-7), the apparatus released at least about 0.42 grams of the substance each day and had an average rate of release of about 0.64 grams/day. During the second week (days 8-14), the apparatus released at least about 0.31 grams of the volatile substance each day and had an average rate of release of about 0.41 grams/day. During the third week (days 15-21), the apparatus released at least about 0.20 grams of the substance each day and had an average rate of release of about 0.25 grams/day. During the fourth week (days 22-28), the apparatus released at least about 0.13 grams of the volatile substance each day and had an average rate of release of about 0.18 grams/day. During the fifth week, (days 29-35), the apparatus released at least about 0.11 grams each day and had an average rate of release of about 0.12 grams/day. During the sixth week (days 36-42), the apparatus released at least about 0.03 grams each day and had an average rate of release of about 0.05 grams/day.

In a third example, an apparatus that is the same as, or similar to, the apparatus used in the first and second examples above was tested to demonstrate the average rate of release of the same oil-based fragrance over 36 days under the same conditions as in Tables 1 and 2. In this example, the starting weight of the apparatus and container was 53.77 grams. As shown in Table 3 below, the weight was measured in daily intervals to show how much substance was diffused into the ambient environment (measured in grams).

TABLE 3

| day | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| output | .88 | .51 | .51 | 1.02 | .83 | .42 | .52 | .46 | .31 | .31 | .65 | .51 | .34 | .43 | .22 |
| day | 16 | 17 | 18 | 19 | 20 | 21 | 22 | 23 | 24 | 25 | 26 | 27 | 28 | 29 | 30 |
| output | .22 | .45 | .25 | .23 | .26 | .24 | .39 | .12 | .15 | .16 | .19 | .23 | .24 | .03 | .09 |
| day | 31 | 32 | 33 | 34 | 35 | 36 | 37 | 38 | 39 | 40 | 41 | 42 | 43 | 44 | 45 |
| output | .07 | .08 | .10 | .11 | .04 | .09 | .02 | .02 | .05 | .05 | .04 | .03 | .04 | .03 | 0 |

In this third test, approximately 0.17 grams of the substance was released in the environment 5 hours after inserting the apparatus into the container. Based on the starting weight and the daily output, the average rate of release over the 45 days was approximately 0.27 grams/day. Over 30 days, the average rate of release was approximately 0.37 grams per day. Further, over the first 34 days, the apparatus diffused at least 0.1 grams of the oil-based fragrance every day. During the first week (days 1-7), the apparatus released at least about 0.42 grams of the oil-based fragrance each day and had an average rate of release of about 0.67 grams/day. During the second week (days 8-14), the apparatus released at least about 0.31 grams of the oil-based fragrance each day and had an average rate of release of about 0.43 grams/day. During the third week (days 15-21), the apparatus released at least about 0.22 grams of the oil-based fragrance each day and had an average rate of release of about 0.27 grams/day. During the fourth week (days 22-28), the apparatus released at least about 0.12 grams of the oil-based fragrance each day and had an average rate of release of about 0.21 grams/day. During the fifth week, (days 29-35), the apparatus released at least about 0.03 grams each day and had an average rate of release of about 0.07 grams/day. During the sixth week (days 36-42), the apparatus released at least about 0.02 grams each day and had an average rate of release of about 0.04 grams/day.

In a fourth example, an apparatus that is the same as, or similar to, the apparatus used in the first, second, and third examples above was tested to demonstrate the average rate of release of the same oil-based fragrance over 36 days under the same conditions as in Tables 1-3. In this example, the starting weight of the apparatus and container was 54.77 grams. As shown in Table 4 below, the weight was measured in daily intervals to show how much substance was diffused into the ambient environment (measured in grams).

TABLE 4

| day | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| output | .72 | .50 | .50 | 1.03 | .83 | .40 | .49 | .43 | .31 | .31 | .62 | .46 | .36 | .37 | .20 |
| day | 16 | 17 | 18 | 19 | 20 | 21 | 22 | 23 | 24 | 25 | 26 | 27 | 28 | 29 | 30 |
| output | .20 | .41 | .20 | .26 | .24 | .22 | .36 | .10 | .14 | .15 | .16 | .17 | .15 | .16 | .15 |
| day | 31 | 32 | 33 | 34 | 35 | 36 | 37 | 38 | 39 | 40 | 41 | 42 | 43 | 44 | 45 |
| output | .11 | .11 | .13 | .17 | .14 | .10 | .06 | .06 | .13 | .09 | .08 | .05 | .06 | .07 | .04 |

In this fourth test, approximately 0.13 grams of the substance was released in the environment 5 hours after inserting the apparatus into the container. Based on the starting weight and the daily output, the average rate of release over the 45 days was approximately 0.27 grams/day. Over 30 days, the average rate of release was approximately 0.35 grams per day. Further, over the first 45 days, the apparatus released at least 0.04 grams of the oil-based fragrance every day. During the first week (days 1-7), the apparatus released at least about 0.4 grams of the oil-based fragrance each day and had an average rate of release of about 0.64 grams/day. During the second week (days 8-14), the apparatus released at least about 0.31 grams of the oil-based fragrance each day and had an average rate of release of about 0.4 grams/day. During the third week (days 15-21), the apparatus released at least about 0.2 grams of the oil-based fragrance each day and had an average rate of release of about 0.25 grams/day. During the fourth week (days 22-28), the apparatus released at least about 0.1 grams of the oil-based fragrance each day and had an average rate of release of about 0.18 grams/day. During the fifth week, (days 29-35), the apparatus released at least about 0.11 grams each day and had an average rate of release of about 0.14 grams/day. During the sixth week (days 36-42), the apparatus released at least about 0.05 grams each day and had an average rate of release of about 0.08 grams/day.

Across the four tests described above (Tables 1-4), the average rate of release was approximately 0.365 grams/day over 30 days and 0.27 grams/day over 45 days. In each of Tables 1-4, there was no substance remaining in the container on or around day 21.

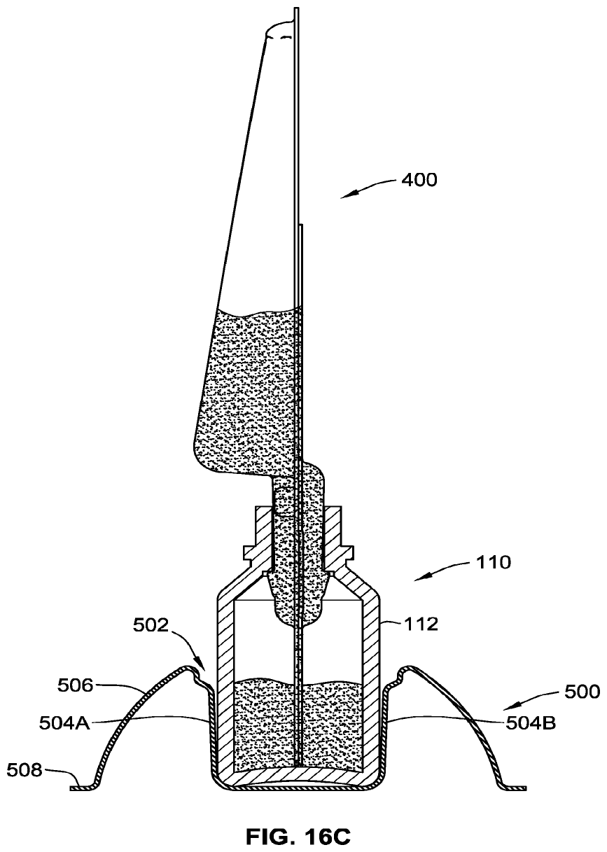

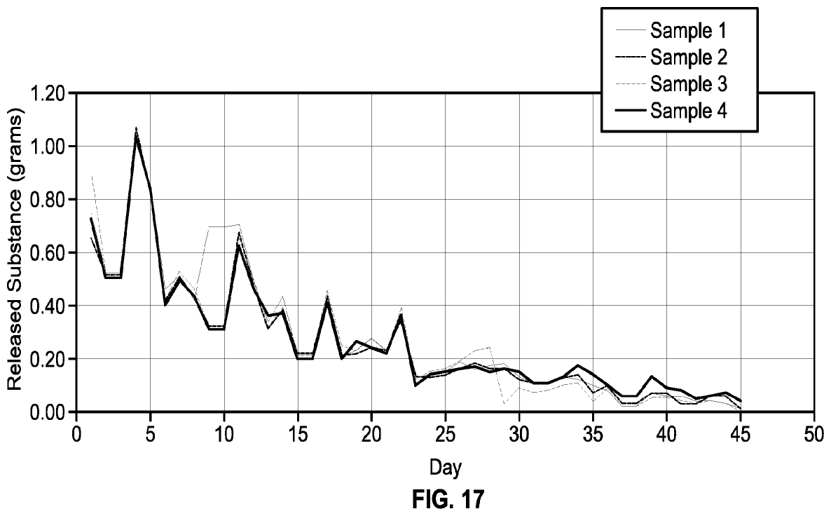

Referring to FIGS. 16A-16B, a base 500 is illustrated. The container 110 described herein can be coupled or secured to the base 500, for example, to aid in preventing the container 110 (and the apparatus 400 if inserted) from tipping or falling over. The base 500 can comprise the same material as the apparatus 400 (e.g., thermoformed molded pulp (Type 3)) or a different material.

The base 500 includes a generally circular opening 502, a plurality of protrusions 504A-504C, a body portion 506, and a flange 508. The opening 502 is sized and shaped for receiving a portion of the body 112 of the container 110 therein, as shown in FIG. 16C. The plurality of protrusions 504A-504C are positioned within the opening 502 and aid in coupling or securing the body 112 of the container 110 within the opening 502 (e.g., via a press or interface fit). As shown, the plurality of protrusions 504A-504C are spaced equally around the circumference of the opening 502. While the plurality of protrusions 504A-504C is shown as including three protrusions, any suitable number of protrusions can be used (e.g., two, four, six, etc.). The body portion 506 extends between the opening 502 and the flange 508. The flange 508 extends along the circumference of the bottom of the base 500 and aids in preventing the base 500 from falling or tipping over.

As shown in FIG. 16C, the body 112 of the container 110 can be inserted into the opening 502 so that the plurality of protrusions 504A-504C engage the surface of the body 112 (e.g., forming a press or interference fit). The container 110 can be coupled to the base 500 when the apparatus 400 is inserted, in which case the base 500 aids in preventing the apparatus 400 and container 110 from tipping or falling over (e.g., if a user accidentally contacts the apparatus 400).

Various systems or kits can be formed including one or more of the components disclosed herein. For example, a first system or kit can include the container 110 (including the substance 116), the apparatus 400, and the base 500. A second system or kit can include the container 110 (including the substance 116) and the apparatus 400. A third system or kit can include a plurality of containers that are the same as, or similar to, the container 110 and the apparatus 400 and optionally the base 500. A third system or kit can include the container 110 (including the substance 116) and the apparatus 100. A fourth system or kit can include the container 110 (including the substance 116), the apparatus 100, and the base 500.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. An apparatus for diffusing a volatile substance to an ambient environment, the apparatus comprising:
a plug configured to be at least partially inserted into an opening of a container containing a volatile substance to aid in at least partially inhibiting the volatile substance from flowing out of the container via the opening; and
a wick portion comprising thermoform molded pulp configured to cause the volatile substance to be drawn into the wick portion through capillary action; and
an emanating portion comprising thermoform molded pulp and coupled to the wick portion such that the volatile substance is drawn into the emanating portion through capillary action, the emanating portion having an exposed surface configured to aid in diffusing the volatile substance into the ambient environment at an average rate of release that is greater than about 0.25 grams of the volatile substance per day over 30 days.

2. The apparatus of claim 1, wherein the thermoformed molded pulp comprises sugarcane bagasse.

3. The apparatus of claim 1, wherein the volatile substance includes an oil-based fragrance.

4. The apparatus of claim 3, wherein the oil-based fragrance comprises 100% oil.

5. The apparatus of claim 4, wherein the average rate of release is greater than about 0.35 grams of the oil-based fragrance per day over 30 days.

6. The apparatus of claim 4, wherein the emanating portion is configured to diffuse at least 0.1 grams of the oil-based fragrance into the ambient environment each day for 30 days.

7. The apparatus of claim 4, wherein the emanating portion is configured to diffuse at least 0.4 grams of the oil-based fragrance into the ambient environment each day during a first week, at least 0.3 grams of the oil-based fragrance into the ambient environment each day during a first week that is immediately subsequent to the first week, at least 0.2 grams of the oil-based fragrance into the ambient environment each day during a third week that is immediately subsequent to the second week, and at least 0.1 grams of the oil-based fragrance into the ambient environment each day during a fourth week that is immediately subsequent to the third week.

8. The apparatus of claim 4, wherein the emanating portion is configured to have an average rate of release of about 0.6 grams/day of the oil-based fragrance during a first week, an average rate of release of about 0.4 grams/day of the oil-based fragrance during a second week that is immediately subsequent to the first week, an average rate of release of about 0.25 grams/day of the oil-based fragrance during a third week that is immediately subsequent to the second week, and an average rate of release of about 0.2 grams/day of the oil-based fragrance during a fourth week that is immediately subsequent to the third week.

9. The apparatus of claim 1, wherein the plug and the wick portion are unitary and/or monolithic and the plug includes a first plug portion and a second plug portion that are moveable relative to one another.

10. The apparatus of claim 9, wherein the wick portion is foldable or includes a hinge configured to permit the first plug portion and the second plug portion of the plug to move relative to one another between an open position and a closed position, wherein the closed position, the first plug portion and the second plug portion are configured to be at least partially inserted into the opening of the container.

11. The apparatus of claim 10, wherein the first plug portion includes a first sealing protrusion configured to engage the container responsive to the plug being inserted into the container to aid in suppressing release of the volatile substance via the opening of the container.

12. The apparatus of claim 11, wherein the second plug portion includes a second sealing protrusion configured to engage the container responsive to the plug being inserted into the container and aid in suppressing release of the volatile substance via the opening of the container.

13. The apparatus of claim 12, wherein the first sealing protrusion and the second sealing protrusion have a tear-drop shape.

14. The apparatus of claim 12, wherein the container has a bottle shape and a screw cap over the opening and a shoulder between the opening and a body of the bottle, the first sealing protrusion and the second sealing protrusion being configured to be inserted into the opening and engage the shoulder inside the container.

15. The apparatus of claim 10, wherein the wick portion further includes a first tongue and a second tongue, wherein in the closed position, the first tongue is coupled to the second tongue.

16. The apparatus of claim 15, wherein the first tongue includes a plurality of apertures and the second tongue includes a plurality of protrusions, wherein in the closed position, the plurality of protrusions engage corresponding ones of the plurality of apertures to aid in coupling the first tongue and the second tongue and inhibiting movement of the first plug portion and the second plug portion relative to one another.

17. The apparatus of claim 16, wherein the plurality of apertures in the first tongue each have a diameter of about 4 mm.

18. The apparatus of claim 1, wherein the emanating portion includes a hole configured to receive therethrough a hanging element for hanging the apparatus as an ornament.

19. The apparatus of claim 1, wherein the emanating portion has a generally triangular shape.

20. The apparatus of claim 1, wherein the emanating portion includes a plurality of grooves extending along a length thereof configured to aid in diffusing the volatile substance into the ambient environment.

21. The apparatus of claim 10, wherein the apparatus has a first length along its longitudinal axis in the open position and a second length along its axis in the closed position, wherein the second length is less than the first length.

22. The apparatus of claim 21, wherein the second length is between about 50% and about 75% of the first length.

23. The apparatus of claim 1, wherein the wick portion, the plug, and the emanating portion are unitary and/or monolithic and each comprise thermoform molded pulp.

24. The apparatus of claim 23, wherein the thermoform molded pulp is of Type 3.

25. The apparatus of claim 1, wherein the wick portion, the plug, and the emanating portion are formed as a unitary, integral piece from a thermoform molded pulp introduced to a mold.

26. The apparatus of claim 1, in combination with the container, the container having a bottle shape and a removable screw cap over the opening, the volatile substance including a fragrance oil, the combination of the apparatus and the container having a weight not exceeding two ounces.

27. The apparatus of claim 26, wherein the emanating portion has a width that is at least 3 times greater than a diameter of the opening of the container.

28. The apparatus of claim 1, in combination with a base including an aperture configured to receive a portion of the container therein.

29. The apparatus of claim 28, wherein the base includes a plurality of protrusions configured to engage a surface of the container to couple the base to the container via an interference fit.

30. The apparatus of claim 1, wherein the apparatus does not include an electronic power supply for aiding in diffusing the volatile substance.
